SEQUENCE LISTING (12 sequences)

SEQ ID NO: 1 - DNA, 35 bp, Artificial sequence
  Primer
gagctcgagttcgccggcatcggcctgaacttctc

SEQ ID NO: 2 - DNA, 33 bp, Artificial sequence
  Primer
ctgctcgagaggagtattacggactgatcgaac

SEQ ID NO: 3 - DNA, 35 bp, Artificial sequence
  Primer
gagtctagattcgccggcatcggcctgaacttctc

SEQ ID NO: 4 - DNA, 33 bp, Artificial sequence
  Primer
ctgtctagaaggagtattacggactgatcgaac

SEQ ID NO: 5 - DNA, 48 bp, Artificial sequence
  Primer
tgagatctggaggctgtcgtcatgagtcgagatgccgacgtcatcgtc

SEQ ID NO: 6 - DNA, 45 bp, Artificial sequence
  Primer
gttgaattcctggtacctcatggggtcttgaaggcgctcgcctca

SEQ ID NO: 7 - DNA, 46 bp, Artificial sequence
  Primer
cggtaccgggagctttgccaatgcatgcagcaaccgccaaggctac

SEQ ID NO: 8 - DNA, 41 bp, Artificial sequence
  Primer
gtgaattccatgctcgagcgggtttagtcacgcctttccag

SEQ ID NO: 9 - DNA, 48 bp, Artificial sequence
  Primer
ccttttggtaccggaggaccgttccatgctgttcaatctcgccatatt

SEQ ID NO: 10 - DNA, 38 bp, Artificial sequence
  Primer
ggggtacctcacgattggctgcgcctgctgcgcaattg

SEQ ID NO: 11 - DNA, 48 bp, Artificial sequence
  Primer
gacggtaccaccggagaaattatgttgtggatttggaatgccctgatc

SEQ ID NO: 12 - DNA, 35 bp, Artificial sequence
  Primer
gtcggtaccttacttcccggatgcgggctcatcct

(19) United States
(12) Patent Application Publication
(10) Pub. No.: US 2008/0193970 A1
(43) Pub. Date: Aug. 14, 2008

(54) Method for Producing Carotenoids and Bacteria Used Therefor

(76) Inventors: Joel Fardoux, Saint Martin de Londres (FR); Eric Giraud, Castelnau Le Lez (FR); Laure Hannibal, Montpellier (FR); Andre Vermeglio, Pertuis (FR)

Correspondence Address: NIXON & VANDERHYE, PC, 901 NORTH GLEBE ROAD, 11TH FLOOR, ARLINGTON, VA, 22203, US

(21) Appl. No.: 10/585,419

(22) PCT Filed: Jan. 10, 2005
(86) PCT No.: PCT/FR2005/000049

§ 371 Date: Jul. 16, 2007

(30) Foreign Application Priority Data
Jan. 9, 2004 (FR) 0400191

(51) Int. Cl. C12P 23/00; C12N 1/20

(52) U.S. Cl. 435/67; 435/252.3

(57) ABSTRACT

The invention concerns a method for synthesizing non-photosynthetic carotenoids using photosynthetic bacteria while producing at least one photosynthetic carotenoid of which one of the synthesis intermediates is lycopene. The inventive method is characterized in that it comprises a number of steps: the deletion and insertion of specific genes; the culturing of bacteria, and; the extraction of carotenoids. The invention also concerns the bacteria or mutants used in the aforementioned method.

The present invention relates to a method for synthesizing carotenoids, in particular non-photosynthetic carotenoids, with photosynthetic bacteria, in particular with Rhodopseudomonas palustris, and to the bacteria used in the method.

More than 600 carotenoids have been identified in nature. They are widespread in a very large number of living organisms, but are synthesized de novo only by a restricted number of organisms (plants, bacteria, yeasts). Some of these carotenoids (lycopene, β-carotene, canthaxanthin, astaxathin) are used intensively and in a very diverse manner in the agrofoods industry for their coloring property (red for lycopene, yellow for β-carotene, orange for canthaxanthin, orangey pink for astaxanthin). The use of astaxanthin and of canthaxanthin is in particular found in aquaculture, for salmon and trout breeding in order to strengthen the orangey pink color of the flesh of the fish, and in aviculture for accentuating the orangey yellow color of the eggs, which, in both cases is pleasing to consumers.

Furthermore, by virtue of their antioxidant properties and their photo-protection role, they are used in the pharmaceutical and cosmetics industry for obtaining, inter alia, protective sun cream and tanning products. In certain countries, an entire series of products derived from canthaxanthin, from β-carotene and from lycopene already exist, which products are sold in beauty and bodybuilding institutes. These carotenoids have also been described as having anticancer properties. In fact, these molecules can reduce the activity of free radicals supposed to contribute to the formation of cancerous cells.

The current synthesis of these carotenoids is mainly carried out chemically. Given that the tendency of the consumer to prefer products of natural origin, there is thus a potential market for carotenoids of plant or microbial origin. Furthermore, it appears that the isomeric configuration of certain carotenoids produced chemically do not correspond to the main isomers found naturally. This difference can prove to be very important since it is liable to result in greater bioassimilation of the carotenoids of natural origin compared with those derived from chemical synthesis.

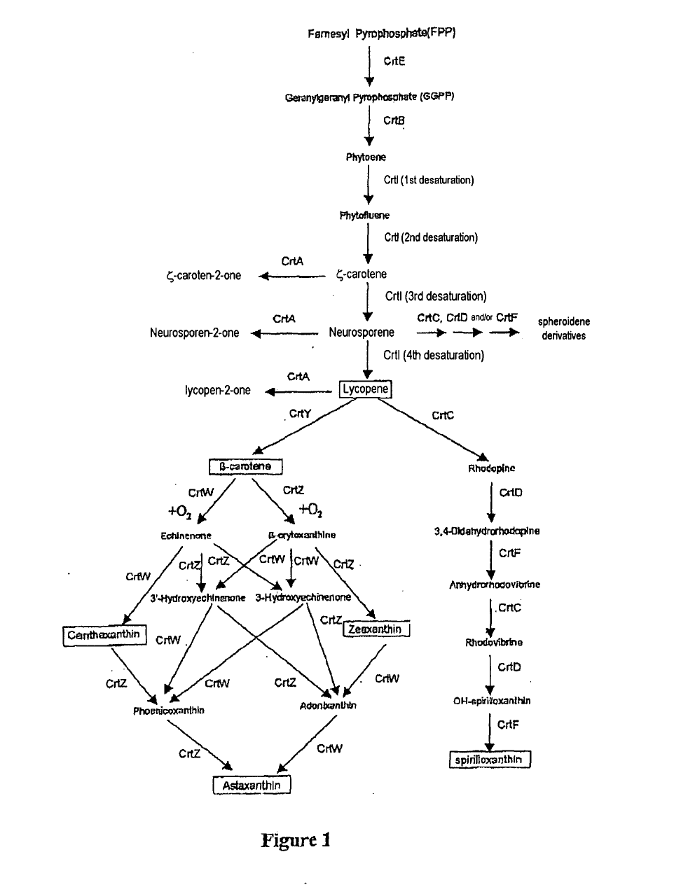

The biosynthetic pathway for these 4 main carotenoids (lycopene, β-carotene, astaxanthin, canthaxanthin) is presented in FIG. 1. The lycopene synthesized by virtue of the successive action of the CrtE, CrtB and CrtI enzymes is an intermediate common to the other 3 carotenoids. The synthesis of β-carotene can thus be carried out from lycopene by virtue of the CrtY enzyme, that of canthaxanthin by virtue of the CrtY and CrtW enzyme and that of astaxanthin by virtue of the CrtY, CrtW and CrtZ enzymes.

Several bacteria have been described for their ability to produce one of these 4 carotenoids (Brevibacterium, Agrobacterium auriantiacum, Erwinia uredovora, Erwina herbicola, Myxococcus xanthus, Flavobacterium, Alcaligenes, Bradyrhizobium, etc.).

In particular, prior studies by the inventors, which were the subject of a filing in the U.S. under the number U.S. 60/297,247, related to the Bradyrhizobium ORS278 strain. These studies demonstrated the existence of a cluster of genes (crtE, crtY, crtI, crtB, crtW) involved in the canthaxanthin biosynthetic pathway. These various genes, once introduced into E. coli, were found to be functional.

With the exception of Bradyrhizobium, which synthesizes a small amount of photosystem, these bacteria are generally non-photosynthetic bacteria which accumulate small amounts of products and which very often have a limited growth rate. The nature of the producer strain thus constitutes a major obstacle for providing a productivity which is sufficiently advantageous for an industrial company.

Moreover, these bacteria generally produce only one of these 4 carotenoids. This means that an industrial company wishing to produce a panel of these 4 main carotenoids must use various bacterial strains which would each require quite specific culture and production conditions. Furthermore, the culturing of various strains on the same site can lead to numerous logistical difficulties related to contamination problems.

Finally, carotenoids are hydrophobic compounds which can be stored in the cell only in a lipophilic environment, generally membrane compartments. The bacteria which produce carotenoids of industrial interest generally have a poorly developed internal membrane. This property, which thus limits the amount of carotenoids that can be accumulated in these cells, constitutes one of the major problems for industrial use of these bacteria.

The studies by the inventors have thus led them to search for a bacterium which can synthesize, according to the culture conditions and/or according to the carotenoid genes introduced into said bacterium, all of these 4 main carotenoids, by overcoming the deficiencies of the other bacteria.

Photosynthetic bacteria exhibit major advantages: a very rapid growth rate and a very high photosynthetic capacity. Carotenoids in photosynthetic bacteria are associated with the photosystems and play an essential role in the functioning thereof. The strong photosynthetic activity is accompanied by the synthesis of a large amount of photosynthetic carotenoids associated with the installation of an abundance of intracytoplasmic membranes constituting a substantial storage zone for the carotenoids.

Nevertheless, these bacteria generally synthesize only one pigment, spirilloxanthin or spheroidene, which at the current time is of no industrial interest.

In order to obtain a microorganism which produces lycopene, β-carotene, canthaxanthin or astaxanthin, the inventors have exploited the potential exhibited by photosynthetic bacteria by diverting the pathway of endogenous synthesis of the photosynthetic carotenoid to the synthesis of non-photosynthetic carotenoids, i.e. not associated with the photosystem, in these bacteria: β-carotene, canthaxanthin or astaxanthin.

The present invention thus relates to a method for synthesizing non-photosynthetic carotenoids chosen from β-carotene, canthaxanthin or astaxanthin, using photosynthetic bacteria which produce at least one photosynthetic carotenoid, one of the synthesis intermediates of which is lycopene, characterized in that it comprises the following steps:
i. deleting, in the bacteria, at least one part of one or more genes involved in the endogenous synthesis pathway which follows that of lycopene, so as to stop said synthesis at the level of lycopene,
ii. inserting the following genes:
   either crtY if the carotenoid to be synthesized is β-carotene,
   or crtY and crtW if the carotenoid to be synthesized is canthaxanthin or β-carotene,
   or crtY, crtZ and crtW if the carotenoid to be synthesized is astaxanthin or β-carotene,
iii. culturing said bacteria thus modified, and
iv. extracting the carotenoid(s) contained in the bacteria.

Surprisingly, it has been noted that a bacterium which synthesizes a photosynthetic carotenoid (i.e. a carotenoid involved in photosynthesis) remains photosynthetic when the pathway of endogenous synthesis of said carotenoid is diverted to the synthesis of non-photosynthetic carotenoids.

This is because the bacterium continues to synthesize, in a residual manner, lycopene, a photosynthetic carotenoid, which allows said bacterium to maintain its photosynthesis activity by replacing in the photosystem the photosynthetic carotenoid synthesized endogenously.

The genes to be at least partially deleted are in general crtC, crtD and/or crtF. Preferably, the crtC and crtD genes will be at least partially deleted. These genes are in fact the first to be used in the synthesis of spirilloxanthin from lycopene (see FIG. 1).

In addition, it may also be necessary to delete crtA in certain bacteria. This will be the case for bacteria which synthesize lycopen-2-one (see FIG. 1).

Preferably, the photosynthetic bacteria are the following: Rubrivivax gelatinosus, Rhodospirillum rubrum, Rhodospirillum moliscianum, Rhodospirillum salinarum, Rhodospirillum mediosalinum, Rhodospirillum sodomense, Rhodocista centenaria, Rhodospira trueperi, Rhodopseudomonas palustris, Rhodopseudomonas acidophila, Rhodopseudomonas julia, Rhodopseudomonas cryptolactis, Rhodomicrobium vannielii, Rhodoplanes roseus, Rhodoplanes elegans, Rhodobium crientis, Rhodobium marinum, etc.

More particularly, the method according to the invention provides culture conditions which ensure production of these various carotenoids at a satisfactory level. In particular, it is a method for synthesizing canthaxanthin or astaxanthin, characterized in that the conditions for culturing the bacteria are sequential and comprise the following steps:
a. culturing said bacteria thus modified firstly under anaerobic conditions under light,
b. then, secondly, under aerobic or under micro-aerobic conditions, in the dark.

In fact, in photosynthetic bacteria, the photosynthetic genes, including the crtE, crtB and crtT genes of the lycopene biosynthetic pathway are under the control of oxygen and of light. In particular, in certain strains, these genes are not expressed in the dark and are inhibited by the presence of oxygen (>8%). This repression by oxygen is the result of a transcription factor called PpsR which, at a high partial oxygen pressure, binds to the promoter regions of the photosynthetic genes, including the crt genes, thus preventing transcription thereof.

On the other hand, the absence of dioxygen is unfavorable to the production of canthaxanthin or of astaxanthin. This is because the enzymatic reaction catalyzed by the CrtW (β-carotene ketolase) or CrtZ (i-carotene hydroxylase) enzymes involved in the biosynthetic pathway for these 2 carotenoids requires oxygen (see FIG. 1). It is thus necessary, in order to allow the synthesis of canthaxanthin and of astaxanthin, to find a compromise in terms of the culture conditions in order to ensure, firstly, the synthesis of the lycopene intermediate and, secondly, its conversion to canthaxanthin or to astaxanthin.

Advantageously, steps a and b of the culture conditions which make it possible to preferentially obtain canthaxanthin or astaxanthin are successively repeated. Such a repetition allows the carotenoids to accumulate in the cells.

Alternatively, the method according to the invention allows the production of β-carotene if the culture conditions are modified. These photosynthetic culturing conditions are then as follows:
a. culturing said bacteria thus modified, under anaerobic conditions under light.

This is because, since the functioning of CrtY enzyme (lycopene cyclase) which allows the conversion of lycopene to β-carotene is not oxygen dependent, the absence of step b of the culture conditions for canthaxanthin or astaxanthin makes it possible to stop the synthesis at the β-carotene stage.

A single bacterium thus makes it possible to synthesize both lycopene and preferentially either β-carotene, or canthaxanthin or astaxanthin, according to the genes introduced and the culture conditions.

Alternatively, the culture conditions which make it possible to preferentially obtain the canthaxanthin or astaxanthin can be carried out under microaerobic conditions under light in a single step.

Under microaerobic conditions, the dioxygen percentage is preferably between 1% and 10%, more particularly 3% to 8%, limits included.

In order to be free of the limitations due to the regulation of the synthesis by dioxygen, the invention also provides a method for synthesizing non-photosynthetic carotenoids chosen from β-carotene, canthaxanthin or astaxanthin, using photosynthetic bacteria which produce at least one photosynthetic carotenoid, one of the synthesis intermediates of which is lycopene, characterized in that the method comprises the following steps:
i. using mutants of photosynthetic bacteria in which the photosynthesis is no longer repressed by dioxygen,
ii. deleting, in the bacteria, at least one part of one or more genes involved in the endogenous synthesis pathway which follows that of lycopene, so as to stop said synthesis at the level of lycopene,
iii. inserting the following genes:
   either crtY if the carotenoid to be synthesized is β-carotene,
   or crtY and crtW if the carotenoid to be synthesized is canthaxanthin or β-carotene,
   or crtY, crtZ and crtW if the carotenoid to be synthesized is astaxanthin or β-carotene,
iv. culturing said bacteria thus modified, under aerobic or microaerobic conditions, in order to synthesize canthaxanthin or astaxanthin, or culturing under anaerobic conditions in order to synthesize β-carotene, and
v. extracting the carotenoid(s) contained in the bacteria.

The mutants in which photosynthesis is no longer repressed by dioxygen are obtained in particular by deletion of the gene encoding the PpsR transcription factor, said factor repressing the expression of certain crt genes at a high partial dioxygen pressure.

Advantageously, the bacteria used in the method are of the Rhodospeudomonas genus, preferably of the species Rhodopseudomonas palustris.

Certain bacteria do not synthesize lycopene, but an intermediate that is further upstream in the carotenoid synthesis pathway. It is then possible to modify these bacteria so as to make them synthesize lycopene.

The photosynthetic bacteria of the method according to the invention are then obtained from photosynthetic bacteria which produce at least one photosynthetic carotenoid, one of the synthesis intermediates of which is phytoene, phytofluene, ζ-carotene or neurosporene, said bacteria having optionally undergone a deletion or disruption of the endogenous crtI gene, followed by insertion of an exogenous crtI gene encoding a phytoene desaturase ensuring 4 successive phytoene desaturation steps.

The bacteria are preferentially: Rhodobacter capsulatus, Rhodobacter veldkampii, Rhodobacter sphaeroides, Rhodobacter azeotoformans, Rhodobacter blasticus, Rhodovulum sulfidophilum, Rhodovulum adriaticum, Rhodovulum euryhalinum, Rhodovulum stricturm, etc.

In such a situation, the genes to be deleted are identical to those mentioned above. In fact, the spheroidene derivatives are obtained from neurosporene (the endogenous crtI encodes a phytoene desaturase ensuring only 3 successive phytoene desaturation steps). The genes encoding the first enzymes subsequently involved are CrtC, CrtD and/or CrtF (see FIG. 1).

In addition, it may also be necessary to delete crtA in certain bacteria. This will be the case for bacteria which synthesize the ζ-caroten-2-one or neurosporen-2-one (see FIG. 1).

Advantageously, the insertion of the crtY, crtZ and/or crtW genes of the method according to the invention is carried out in the zone of the genes at least partially deleted.

Alternatively, the crtY, crtZ and/or crtW genes can also be inserted into a plasmid.

The invention also covers the photosynthetic bacteria which produce, in an alternating or concomitant manner, at least lycopene, β-carotene and canthaxanthin or astaxanthin, characterized in that said bacteria can be obtained by means of the method according to the invention.

In particular, these photosynthetic bacteria which synthesize at least one carotenoid, the synthesis intermediate of which is lycopene, are characterized in that they comprise the following mutations:
i. deletion of at least one part of one of more genes involved in the endogenous synthesis pathway which follows that of lycopene, so as to stop said synthesis at the level of lycopene,
ii. insertion of the following genes:
   either crtY if the carotenoid to be synthesized is β-carotene,
   or crtY and crtW if the carotenoid to be synthesized is canthaxanthin or β-carotene,
   or crtY, crtZ and crtW if the carotenoid to be synthesized is astaxanthin or β-carotene.

Alternatively, it is a mutant of photosynthetic bacteria synthesizing at least one carotenoid, the synthesis intermediate of which is lycopene, in which the synthesis of the photosystem is no longer repressed by dioxygen, producing canthaxanthin or astaxanthin, characterized in that it comprises the following mutations:
i. deletion of at least one part of one or more genes involved in the endogenous synthesis pathway which follows that of lycopene, so as to stop said synthesis at the level of lycopene,
ii. insertion of the following genes:
   either crtY if the carotenoid to be synthesized is β-carotene,
   or crtY and crtW if the carotenoid to be synthesized is canthaxanthin or β-carotene,
   or crtY, crtZ and crtW if the carotenoid to be synthesized is astaxanthin or β-carotene.

These bacteria or mutants can be obtained from bacteria or from mutants which produce at least one carotenoid, one of the synthesis intermediates of which is phytoene, phytofluene, ζ-carotene or neurosporene, said bacteria or mutants having optionally undergone a deletion or disruption of the endogenous crtI gene, followed by insertion of an exogenous crtI gene encoding a phytoene desaturase ensuring 4 successive phytoene desaturation steps.

Surprisingly, it has been noted that these photosynthetic bacteria or photosynthetic bacterial mutants, although their pathway for synthesis of the endogenous photosynthetic carotenoid has been diverted to the synthesis of a non-photosynthetic carotenoid of interest, maintain their photosynthetic activity.

Other characteristics and advantages of the invention will emerge in the examples with references to the following figures:

FIG. 1 represents the scheme summarizing the various carotenoids targeted by the invention,

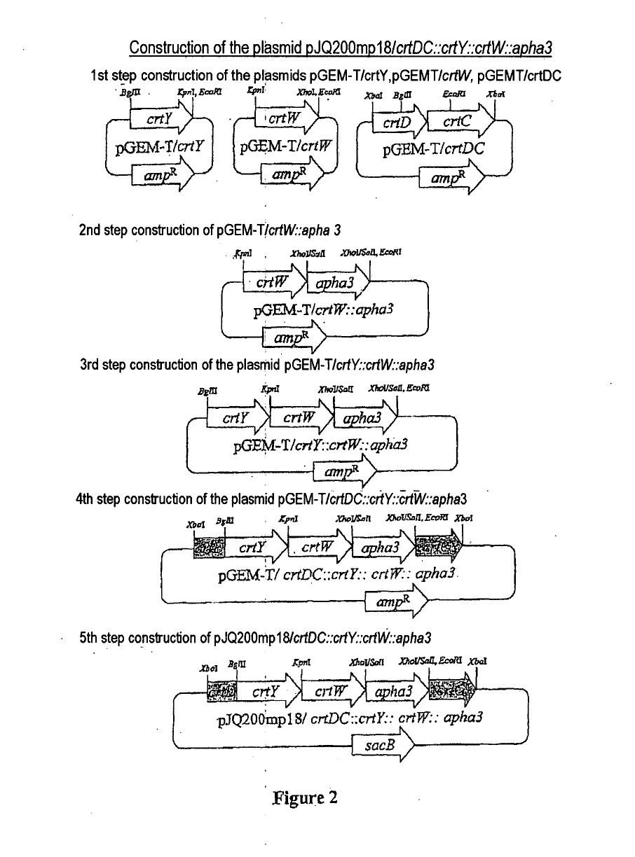

FIG. 2 shows the scheme summarizing the strategy for constructing the plasmid pJG200mp18/crtDC::crtY::crtW::apha3,

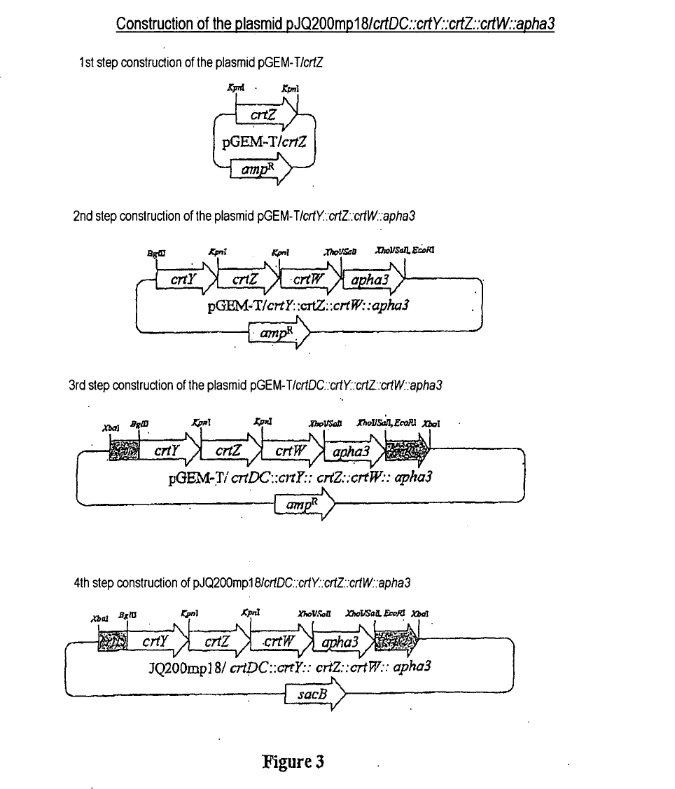

FIG. 3 shows the scheme summarizing the strategy for constructing the plasmid pJQ200mp18/crtDC::crtY::crtZ::crtW::apha 3, and

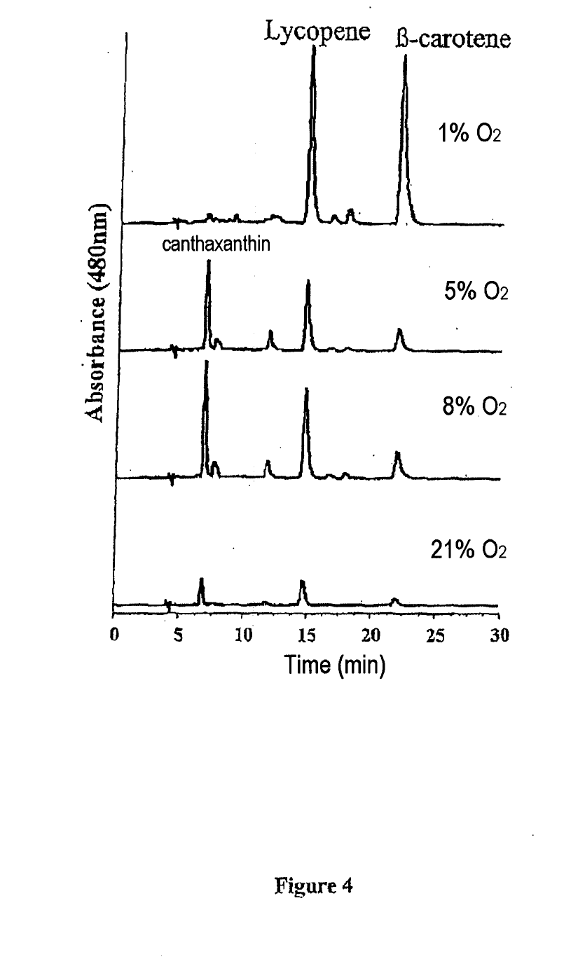

FIG. 4 is an HPLC analysis of the carotenoids synthesized by the R. palustris mutant strain CEA001ΔcrtDC::crtY::crtW under various culture conditions.

### EXAMPLE 1

### Construction of a Mutant Strain of Rhodopseudomonas palustris Producing Lycopene

The sequence of the R. palustris crt gene cluster involved in the synthesis of spirilloxanthin is accessible on the internet at the following address http://www.ncbi.nlm.nih.gov (Acc Number: BX572597).

As in Bradyrhizobium ORS278, the crtE, crtI, crtB, crtC, crtD and crtF genes are present and organized in 3 operons.

The general strategy for constructing this mutant strain will consist in disrupting the crtD and crtC genes by inserting therein a cassette encoding a kanamycin resistance gene (apha3 gene).

1st Step: Isolation and Cloning of the R. palustris crtDC Genes:

A region corresponding to a part of the R. palustris crtDC genes was amplified using the pair of primers:

| | |
|---|---|
| crtD.R.palu.XhoI.f: | |
| GAGCTCGAGTTCGCCGGCATCGGCCTGAACTTCTC | (SEQ ID No. 1) |
| CrtC.R.palu.XhoI.r: | |
| CTGCTCGAGAGGAGTATTACGGACTGATCGAAC | (SEQ ID No. 2) |

The primers were designed so as to insert an XhoI restriction site on either side of the PCR product.

The amplifications were carried out with a high fidelity DNA polymerase sold by Invitrogen® (Platinum Pfx DNA polymerase).

The amplification conditions are as follows: after an initial DNA denaturation step at 95° C. for 5 min, 35 PCR cycles are carried out (94° C. for 15 s then 55° C. for 30 s then 68° C. for 3 min), followed by a final elongation step (68° C. for 7 min) during which 1 μl of conventional Taq polymerase (GoTaq, Promega®) is added to the PCR tube in order to insert a deoxyadenosine residue at the 3′ ends of the amplification fragments (this step is essential for allowing cloning into the vector pGEM-T subsequently used).

The PCR product thus obtained is cloned into the cloning vector pGEM-T (sold by the company Promega®) according to the supplier's protocol. The plasmid obtained is called pGEM-T/crtDC.XhoI.

2nd step: Construction of the Plasmid pJQ200mp18/crtDC

The region corresponding to the R. palustris crtDC genes is released by XhoI digestion of the plasmid pGME-T/crtDC.XhoI, and then ligated into the suicide vector pJQ200mp18 linearized by SalI digestion. This plasmid contains the sacB suicide gene which makes it possible to select on sucrose the clones for which a double crossing over event has indeed taken place (Quandt, J. & Hynes, M. F. “Versatile suicide vectors which allow direct selection for gene replacement in gram-negative bacteria”: Gene 127, 15-21, 1993).

3rd Step: Construction of the Plasmid pJQ200mp18/crtDC:aphA-3

The aphA-3 gene encoding kanamycin resistance is released from the plasmid pUC4K sold by the company Amersham® by digestion with the SalI enzyme. It is then inserted into the plasmid pJQ200mp18/crtDC linearized with SalI. It should be noted that the latter SalI digestion results in a deletion of a 618 bp fragment corresponding to the 5′ end of the crtD gene and the 3′ end of the crtC gene.

The plasmid pJQ200mp18/crtDC::aphA-3 is finally transferred by electroporation into the conjugative Escherichia coli S17.1 strain.

4th Step: Construction of a Mutant Strain of R. palustris Containing the apha3 Cassette in the crtD, crtC Genes

The previous plasmid pJQ200mp18/crtDC::aphA-3 is delivered into the wild-type R. palustris strain (CEA001) by conjugation with the previous E. coli S17.1 strain containing the construct. The conjugant clones are selected on Hutner medium (R. K. Clayton “Towards the isolation of a photochemical reaction center in Rhodopseudomonas sphaeroides”; Biochim. Biophys. Acta 75:312-318, 1963) containing 150 μg/ml of kanamycin and 50 μg/ml of carbenicillin. The double recombinant clones are selected after subculturing of the conjugant clones on Hutner medium containing 5% sucrose and kanamycin (150 μg/ml). A PCR verification is carried out on several recombinant clones in order to be sure of the insertion of the apha3 cassette into the R. palustris crtD and crtC genes.

The production of a large amount of lycopene was noted in the bacteria thus mutated. Moreover, the photosynthetic nature of the bacterium is conserved.

### EXAMPLE 2

### Construction of a Mutant Strain of Rhodopseudomonas palustris Producing β-Carotene or Canthaxanthin

### Construction Scheme

The strategy for constructing this mutant strain is summarized in FIG. 2.

In this figure, the legends of the steps are as follows:

1st step: Amplification of the crtY and crtW genes of Bradyrhizobium ORS278, and also of the crtDC genes of R. palustris. Cloning the PCR products into the plasmid p-GEMT (Promega).
2nd step: Release, by SalI, of the apha3 cassette from the plasmid puc4K sold by Amersham. Insertion at the unique XhoI site present downstream of crtW.
3rd step: Release of the crtW and apha3 genes with KpnI/EcoRI. Insertion into the plasmid pGEM-T/crtY linearized with the same enzymes.
4th step: Release of the crtY, crtW and apha3 genes with BglII/EcoRI. Insertion into the plasmid PGEM-T-crtDC linearized with the same enzymes.
5th step: Release of the crtDC, crtY, crtW and aph3 genes with XbaI. Insertion into the plasmid pJQ200mp18 linearized with the same enzyme.

### A. Construction Strategy

The overall strategy for constructing a mutant strain of R. palustris producing either β-carotene or canthaxanthin consisted in disrupting the R. palustris crtD, crtC genes by inserting therein the Bradyrhizobium ORS278 crtY and crtW genes and also an aphA-3 gene encoding kanamycin resistance.

Depending on the oxygenation conditions of the culture (see example 3), the CrtW enzyme which converted β-carotene to canthaxanthin is or is not functional, which results, depending on choice, in an accumulation of β-carotene or of canthaxanthin.

1st Step: Isolation and Cloning of the R. palustris crtDC and Bradyrhizobium ORS278 crtY and crtW Genes:

A region corresponding to a part of the R. palustris crtDC genes was amplified using the pair of primers:

| | |
|---|---|
| crtD.R.palu.XbaI.f: | |
| GAGTCTAGATTCGCCGGCATCGGCCTGAACTTCTC | (SEQ ID No. 3) |
| CrtC.R.palu.XbaI.r: | |
| CTGTCTAGAAGGAGTATTACGGACTGATCGAAC. | (SEQ ID No. 4) |

The primers were designed so as to insert an XbaI restriction site on either side of the PCR product.

The Bradyrhizobium ORS278 crtY gene was amplified using the following pair of primers:

crtY.278.f:

(SEQ ID No. 5)

TGAGATCTGGAGGCTGTCGTCATGAGTCGAGATGCCGACGTCATCGTC
crtY.278.r:

(SEQ ID No. 6)

GTTGAATTCCTGGTACCTCATGGGGTCTTGAAGGCGTCGCCTCA.

The primers were designed so as to insert an RBS ribosomal binding site and a BglII restriction site in the 5′ position and also a KpnI and EcoRI restriction site in the 3′ position of the PCR product.

The Bradyrhizobium ORS278 crtW gene was amplified using the following pair of primers:

crtW.ORS278.f:

(SEQ ID No. 7)

CGGTACCGGGAGCTTTGCCAATGCATGCAGCAACCGCCAAGGCTAC
crtW.ORS278.r:

(SEQ ID No. 8)

GTGAATTCCATGCTCGAGCGGGTTTAGTCACGCCTTTCCAG.

The primers were designed so as to insert an RBS ribosomal binding site and a Kpn I restriction site in the 5′ position and also an Xho I and EcoR I restriction site in the 3′ position of the PCR product.

The amplifications were carried out with a high fidelity DNA polymerase sold by Invitrogen® (Platinum Pfx DNA polymerase).

The amplification conditions are as follows: after an initial DNA denaturation step at 95° C. for 5 min, 35 PCR cycles are carried out (94° C. for 15 s then 55° C. for 30 s then 68° C. for 3 min), followed by a final elongation step (68° C. for 7 min) during which 1 μl of conventional Taq polymerase (GoTaq, Promega®) is added to the PCR tube in order to insert a deoxyadenosine residue at the 3′ ends of the amplification fragments (this step is essential for allowing cloning into the vector pGEM-T subsequently used).

The 3 PCR products thus obtained are cloned into the cloning vector PGEM-T (sold by the company Promega®) according to the supplier's protocol. The plasmids obtained are called pGEM-T/crtDC.XbaI; pGEM-T/crtY; pGEM-T/crtW.

2nd Step: Construction of the Plasmid pGEM-T/crtW::aphA-3

The aphA-3 gene encoding kanamycin resistance is released from the plasmid pUC4K sold by the company Amersham by digestion with the SalI enzyme. It is then inserted into the plasmid pGEM-T/crtW linearized with XhoI.

3rd Step: Construction of the Plasmid pGEM-T/crtY::crtW::aphA-3

The previous construct containing the crtW and aphA-3 genes is released by KpnI/EcoRI double digestion and then inserted into the plasmid pGEM-T/crtY linearized with the same pair of restriction enzymes.

4th Step: Construction of the Plasmid pGEM-T/crtDC::crtY:: crtW::aphA-3

The previous construct containing the crtY, crtW and aphA-3 genes is released by BglII/EcoRI double digestion and then inserted into the plasmid pGEM-T/crtDC linearized with the same pair of restriction enzymes. The digestion of pGEM-T/crtDC with BglII/EcoRI results in a deletion of a 1232 bp fragment corresponding to a considerable part of the crtD and crtC genes.

5th Step: Construction of the Plasmid pJQ200mp18/crtDC::crtY::crtW::aphA-3

The previous construct containing the crtDC, crtY, crtW and aphA-3 genes is released by XbaI digestion and then inserted into the suicide plasmid pJQ200mp18 linearized with XbaI. The plasmid pJQ200mp18/crtDC:::crtY::crtZ::crtW::aphA-3 is finally transferred by electroporation into the conjugative Escherichia coli S17.1 strain.

6th Step: Construction of a Mutant Strain of R. palustris Containing the Bradyrhizobium ORS278 crtY and crtW Genes

The previous plasmid pJQ200mp18/-crtDC::crtY::crtW::aphA-3 is delivered into the R. palustris CEA001 strain by conjugation with the previous E. coli S17.1 strain containing the construct. The conjugant clones are selected on Hutner medium containing 150 μg/ml of kanamycin and 50 μg/ml of carbenicillin. The double recombinant clones are selected after subculturing of the conjugant clones on Hutner medium containing 5% sucrose and kanamycin (150 μg/ml). A PCR verification is carried out on several recombinant clones in order to ensure that the crtY and crtW genes have indeed been inserted into the crtD and crtC genes. A mutant is selected for the remainder of the experiments: R. palustris CEA001ΔcrtDC::crtY::crtW.

B. Analysis of the Carotenoid Produced by the R. palustris Mutant Strain CEA001ΔcrtDC::crtY::crtW—Trial Under Various Culture Conditions

In order to determine the potential of the R. palustris mutant strain CEA001ΔcrtDC::crtY::crtW to overproduce β-carotene and canthaxanthin, a series of preliminary trials was carried out in which various culture conditions were tested.

A: 120 ml flask containing 100 ml of Hutner medium, 30° C.—degassed so as to remove all traces of oxygen, and placed under a 100 watt incandescence lamp (conditions light, anaerobiosis);
B: 250 ml flask filled with 50 ml of Hutner medium, 30° C.—shaking at 170 rpm—placed in an atmosphere where the oxygen content can be adjusted to 1, 5, 8% (conditions light, microaerobiosis) or 21% (conditions light, aerobiosis);
C: after culturing under anaerobic conditions for 2 days according to the conditions A, 50 ml are transferred into a baffled Erlenmeyer flask and then shaken at 170 rpm in the dark overnight in order to highly oxygenate the culture.

After culturing for 3 days under the various conditions tested, the carotenoids produced are analyzed by HPLC. The results obtained are given in table 1 below.

TABLE 1
Production of carotenoids according to the
culture conditions in the R. palustris mutant
CEA001ΔcrtDC::crtY::crtW

| | | Lycopene | β-carotene | Canthaxanthin |
|---|---|---|---|---|
| Conditions | OD650 | mg/l | mg/l | mg/l |
| A | 3.7 | 2.7 | 4.7 | 0 |
| B 1% 0₂ | 2.9 | 0.85 | 1.13 | 0.1 |
| B 5% O₂ | 2 | 0.35 | 0.19 | 0.32 |
| B 85% O₂ | 2.4 | 0.46 | 0.16 | 0.41 |
| B 21% O₂ | 1.8 | 0.12 | 0.05 | 0.07 |
| C | 3.7 | 2.4 | 3.8 | 0.8 |

It clearly appears that the aeration and light conditions have very substantial effects on the production of the carotenoids and the growth of the strain. Large amounts of β-carotene can be obtained under anaerobic conditions under light (4.7 mg/l); on the other hand, only low concentrations of carotenoids (lycopene and β-carotene) are obtained when the bacterium is cultured under aerobic conditions (21%) because of the repression of the synthesis of the photosynthetic apparatus by oxygen. The presence of large amounts of β-carotene under anaerobic conditions with light clearly shows that the crtY gene of ORS278 which allows the conversion of lycopene to β-carotene is perfectly functional. On the other hand, an absence of production of canthaxanthin is observed under these same conditions. This results from the fact that the CrtW enzyme which allows the conversion of β-carotene to canthaxanthin is an oxygenase which thus needs oxygen in order to function. In agreement with this explanation, the concentration of canthaxanthin gradually increases (from 0.32 to 0.41 mg/l) when the oxygen content goes from 5 to 8% with a concomitant decrease in β-carotene (table I). This experiment demonstrates, firstly, the need for the presence of oxygen for the formation of canthaxanthin from β-carotene and, secondly, that the CrtW enzyme of ORS278 is also functional in this mutant. The conversion of β-carotene to canthaxanthin according to the oxygen content is also demonstrated by the chromatogram shown in FIG. 4. Another argument in favor of a conversion of β-carotene to canthaxanthin in an equimolar manner in the presence of oxygen is presented under the conditions C, i.e. by oxygenating the culture after growth under anaerobic conditions, where it is possible to convert a part of this β-carotene to canthaxanthin.

These first trials clearly show that it is possible to produce canthaxanthin in R. palustris. Moreover, by adjusting the aeration conditions, the amount of β-carotene can be converted to canthaxanthin should be close to 5 mg/l, i.e. 6 times more than the amount of canthaxanthin (0.8 mg/l) obtained in liquid culture with the Bradyrhizobium ORS278 strain. Furthermore, the culturing time was more than halved, i.e. the productivity was increased by the same ratio.

### EXAMPLE 3

### Strategy for Constructing a Mutant Strain of Rhodopseudomonas palustris Producing Astaxanthin

### Construction Scheme

The strategy for constructing this mutant strain is summarized in FIG. 3.

In this figure, the legends of the steps are as follows:

1st step: Amplification of the Pseudomonas putida crtZ gene. Cloning of the PCR product in the plasmid p-GEMT (Promega).
2nd step: Release of the crtZ gene with KpnI. Insertion into the unique KpnI site of the plasmid pGEM-T/crtY::crtW::apha3.
3rd step: Release of the crtY, crtZ, crtW and apha3 genes with BglII/EcoRI. Insertion into the plasmid pGEM-T/crtDC linearized with the same enzymes.
4th step: Release of the crtDC, crtY, crtZ, crtW and apha3 genes with XbaI. Insertion into the plasmid pJQ200mp18 linearized with the same enzyme.

### Construction

The general strategy consists in inserting the Pseudomonas putida gene into the previously constructed mutant strain of R. palustris containing the Bradyrhizobium ORS278 crtY and crtW genes. The insertion is carried out into the unique KpnI restriction site present between the crtY and crtW genes.

1st Step: Construction of the Plasmid pGEM-T/crtZ

The Pseudomonas putida genome was entirely sequenced.

The sequence of the crtZ gene is available in the gene banks (Genbank ACC: NC_{—}00297). The gene is amplified from the genomic DNA of Pseudomonas putida using the pair of primers:

crtZ.Ps.putida.f:

(SEQ ID No. 9)

CCTTTTGGTACCGGAGGACCGTTCCATGCTGTTCAATCTCGCCATATT
crtZ.Ps.putida.r:

(SEQ ID No. 10)

GGGGTACCTCACGATTGGCTGCGCCTGCTGCGCAATTG.

The primers were designed so as to insert an RBS ribosomal binding site in the 5′ position and a KpnI restriction site in the 5′ position and in the 3′ position of the PCR product.

The amplification is carried out with a high fidelity DNA polymerase sold by Invitrogen® (Platinum Pfx DNA polymerase).

The amplification conditions are as follows: after an initial DNA denaturation step at 95° C. for 5 min, 35 PCR cycles are carried out (94° C. for 15 s, then 55° C. for 30 s, then 68° C. for 1 min), followed by a final elongation step (68° C. for 7 min), during which 1 μl of conventional Taq polymerase (GoTaq, Promega®) is added to the PCR tube in order to insert a deoxyadenosine residue at the 3′ ends of the amplification fragments (this step is essential for allowing cloning into the vector pGEM-T subsequently used).

The PCR product thus obtained is cloned into the cloning vector pGEM-T according to the supplier's protocol. The plasmid obtained is called pGEM-T/crtZ.

2nd Step: Construction of the Plasmid pGEM-T/crtDC::crtY::crtZ::crtW::aphA-3

The construct previously obtained, pGEM-T/crtDC::crtY::crtW::aphA-3 containing the Bradyrhizobium ORS278 crtW and crtY genes and the aphA-3 kanamycin resistance gene inserted into the R. palustris crtDC genes, is linearized with the KpnI enzyme, the restriction site of which is located between the crtW and crtY genes. The crtZ gene cloned into the vector pGEM-T/crtZ is released with KpnI, and then inserted by ligation into the linearized previous construct.

3rd Step: Construction of the Plasmid pJQ200mp18/crtDC::crtY::crtZ::crtW::aphA-3

The previous construct containing the crtDC, crtY, crtZ, crtW and aphA-3 genes is released by XbaI digestion and then inserted by ligation into the suicide plasmid pJQ200mp18 linearized with XbaI. The plasmid pJQ200mp18/crtDC::crtY::crtZ::crtW::aphA-3 is transferred by electroporation into the conjugative Escherichia coli S17.1 strain.

4th Step: Construction of a Mutant Strain of R. palustris CEA001 Containing the Bradyrhizobium ORS278 crtY and crtW and Pseudomonas putida crtZ Genes

The previous plasmid pJQ200mp18/crtDC::crtY::crtZ::crtW::aphA-3 is delivered into the R. palustris strain CEA001 by conjugation with the previous E. coli S17.1 strain contained in the construct. The conjugant clones are selected on Hutner medium containing 150 μg/ml of kanamycin and 50 μg/ml of carbenicillin. The double recombinant clones are selected after subcloning of the conjugant clones on Hutner medium containing 5% sucrose and kanamycin (150 μg/ml). A PCR verification is carried out on several recombinant clones in order to ensure that the crtY, crtZ and crtW genes have indeed been inserted into the crtD and crtC genes.

Under microaerobic conditions, the production of astaxanthin was observed in the bacteria thus mutated.

### EXAMPLE 4

### Alternative Strategy for Constructing a Mutant Strain of Rhodopseudomonas palustris Producing Astaxanthin

Compared with the construction strategy described in example 3, the crtZ gene essential for producing astaxanthin was isolated from a strain of Erwinia uredovora and not Pseudomonas putida. The construction strategy remains identical to that described in example 3, with the exception of the pair of primers used for isolating the crtZ gene by PCR.

New primers:

crtZ.Er.uredovora.f:
GACGGTACCACCGGAGAAATTATGTTGTGGATTTGGAATGCCCTGATC
crtZ.crtZ.Er.uredovora.r:
GTCGGTACCTTACTTCCCGGATGCGGGCTCATCCT.

The primers used were also defined so as to insert an RBS binding site in the 5′ position and a KpnI restriction site in the 5′ position and in the 3′ position of the PCR.

After construction, the ability of the R. palustris mutant strain CEA001ΔcrtCD::crtY::crtZ::crtW::apha3 to produce astaxanthin was verified by HPLC after culturing under various oxygen conditions [250 ml flask filled with 50 ml of Hutner medium, 30° C.—shaking at 170 rpm—placed in an atmosphere where the oxygen content can be adjusted to 1, 5, 8% (conditions light, microaerobiosis) or 21% (conditions light, aerobiosis)—culturing time 3 days].

The results obtained are given in the table below.

TABLE 2
Production of carotenoids according to the culture conditions in the
R. palustris mutant CEA001ΔcrtCD::crtY::crtZ::crtW::apha3

| | | Lycopene | β-carotene | Zeaxanthin | Asthaxanthin | Canthaxanthin |
|---|---|---|---|---|---|---|
| Conditions | OD 650 | mg/l | mg/l | mg/l | mg/l | mg/l |
| 1R O₂ | 5.24 | 7.197 | 1.805 | 0.633 | — | — |
| 5% O₂ | 2.57 | 0.483 | 0.123 | — | 0.76 | 0.19 |
| 8% O₂ | 1.83 | 0.133 | 0.043 | — | 0.146 | 0.043 |
| 21% O₂ | 1.57 | 0.103 | 0.103 | — | 0.32 | — |

As already described, a very substantial effect of the oxygen content on the amounts and the varieties of carotenoids produced is noted. When the oxygen content is fixed at 5%, the major carotenoid produced is astaxanthin. These trials thus show that it is possible to overproduce astaxanthin in R. palustris.

1. A method for synthesizing non-photosynthetic carotenoids chosen from β-carotene, canthaxanthin or astaxanthin, using photosynthetic bacteria which produce at least one photosynthetic carotenoid, one of the synthesis intermediates of which is lycopene, characterized in that it comprises the following steps:
i. deleting, in the bacteria, at least one part of one or more genes involved in the endogenous synthesis pathway which follows that of lycopene, so as to stop said synthesis at the level of lycopene,
ii. inserting the following genes:
either crtY if the carotenoid to be synthesized is β-carotene,
or crtY and crtW if the carotenoid to be synthesized is canthaxanthin or β-carotene,
or crtY, crtZ and crtW if the carotenoid to be synthesized is astaxanthin or β-carotene,
iii. culturing said bacteria thus modified, and
iv. extracting the carotenoid(s) contained in the bacteria.

2. The method as claimed in claim 1, for synthesizing canthaxanthin or astaxanthin, characterized in that the culturing conditions are sequential and comprise the following steps:
c. culturing said bacteria thus modified firstly under anaerobic conditions under light,
d. then, secondly, under aerobic conditions, in the dark.

3. The method as claimed in claim 1, for synthesizing β-carotene, characterized in that the culturing conditions are as follows:
b. culturing said bacteria thus modified, under anaerobic conditions under light.

4. The method as claimed in claim 2, characterized in that steps a and/or b are carried out under microaerobic conditions under light.

5. The method as claimed in claim 4, characterized in that, under microaerobic conditions, the dioxygen percentage is between 1% and 10%, preferably 3% to 8%, limits included.

6. The method as claimed in claim 2, characterized in that steps a and b are successively repeated.

7. A method for synthesizing non-photosynthetic carotenoids chosen from β-carotene, canthaxanthin or astaxanthin, using photosynthetic bacteria which produce at least one photosynthetic carotenoid, one of the synthesis intermediates of which is lycopene, characterized in that the method comprises the following steps:
i. using mutants of photosynthetic bacteria in which the photosynthesis is no longer repressed by dioxygen,
ii. deleting, in the bacteria, at least one part of one or more genes involved in the endogenous synthesis pathway which follows that of lycopene, so as to stop said synthesis at the level of lycopene,
iii. inserting the following genes:
either crtY if the carotenoid to be synthesized is β-carotene,
or crtY and crtW if the carotenoid to be synthesized is canthaxanthin or β-carotene,
or crtY, crtZ and crtW if the carotenoid to be synthesized is astaxanthin or β-carotene,
iv. culturing said bacteria thus modified, under aerobic or microaerobic conditions, in order to synthesize canthaxanthin or astaxanthin, or culturing under anaerobic conditions in order to synthesize β-carotene, and
v. extracting the carotenoid(s) contained in the bacteria.

8. The method as claimed in claim 7, characterized in that the mutants in which the photosynthesis is no longer repressed by dioxygen are obtained by deletion of the gene encoding the PpsR transcription factor.

9. The method as claimed in claim 1, characterized in that the bacteria are of the Rhodopseudomonas genus, preferably of the species Rhodopseudomonas palustris.

10. The method as claimed in claim 1, characterized in that said photosynthetic bacteria which produce at least one photosynthetic carotenoid, one of the synthesis intermediates of which is lycopene, are obtained from photosynthetic bacteria which produce at least one photosynthetic carotenoid, one of the synthesis intermediates of which is phytoene, phytofluene, ζ-carotene or neurosporene, said bacteria having optionally undergone a deletion or disruption of the endogenous crtI gene, followed by insertion of an exogenous crtI encoding a phytoene desaturase ensuring 4 successive phytoene desaturation steps.

11. The method as claimed in claim 1, characterized in that the insertion of the crtY, crtZ and/or crtW genes is carried out in the zone of the genes at least partially deleted.

12. A photosynthetic bacterium which produces, in an alternating or concomitant manner, at least lycopene, β-carotene and canthaxanthin or astaxanthin, characterized in that said bacterium can be obtained by means of the method as claimed in claim 1.

13. A photosynthetic bacterium, characterized in that it is obtained according to the following method:
i. culturing a photosynthetic bacterium which synthesizes at least one photosynthetic carotenoid, the synthesis intermediate of which is lycopene,
ii. deleting at least a part of one or more genes involved in the endogenous synthesis pathway which follows that of lycopene, so as to stop said synthesis at the level of lycopene,
iii. inserting the following genes:
either crtY if the carotenoid to be synthesized is β-carotene,
or crtY and crtW if the carotenoid to be synthesized is canthaxanthin or β-carotene,
or crtY, crtZ and crtW if the carotenoid to be synthesized is astaxanthin or β-carotene.

14. A mutant of a photosynthetic bacterium, characterized in that it is obtained according to the following method:
i. using a mutant of a photosynthetic bacteria synthesizing at least one photosynthetic carotenoid, the synthesis intermediate of which is lycopene, in which the photosynthesis is no longer repressed by dioxygen, producing canthaxanthin or astaxanthin,
ii. deleting at least a part of one or more genes involved in the endogenous synthesis pathway which follows that of lycopene, so as to stop said synthesis at the level of lycopene,
iii. inserting the following genes:
either crtY if the carotenoid to be synthesized is β-carotene,
or crtY and crtW if the carotenoid to be synthesized is canthaxanthin or β-carotene,
or crtY, crtZ and crtW if the carotenoid to be synthesized is astaxanthin or β-carotene.

15. The bacterium as claimed in claim 12, characterized in that it is respectively obtained from a bacterium or from a mutant which produces at least one photosynthetic carotenoid, one of the synthesis intermediates of which is phytoene, phytofluene, ζ-carotene or neurosporene, said bacterium or mutant having optionally undergone a deletion or disruption of the endogenous crtI gene, followed by insertion of an exogenous crtI gene encoding a phytoene desaturase ensuring 4 successive phytoene desaturation steps.
